(19) United States
(12) Patent Application Publication
(10) Pub. No.: US 2026/0021284 A1
(43) Pub. Date: Jan. 22, 2026

(54) SELECTIVE MICROSPHERE DELIVERY SYSTEM WITH SPACE EFFICIENT TUBING GEOMETRY

(71) Applicant: Boston Scientific Scimed, Inc., Maple Grove, MN (US)

(72) Inventors: Scott McGhee, Ottawa (CA); Kianna Y. Elahi-Gedwillo, Minneapolis, MN (US); Jonathan Nath, Inver Grove Heights, MN (US); Logan Ernster, Saint Paul, MN (US); Jacob Stoesz, Golden Valley, MN (US)

(21) Appl. No.: 19/270,057

(22) Filed: Jul. 15, 2025

Related U.S. Application Data
(60) Provisional application No. 63/673,064, filed on Jul. 18, 2024.

(51) Int. Cl. A61M 39/08; A61M 39/00; A61M 39/22

(52) U.S. Cl. CPC A61M 39/08; A61M 39/22; A61M 2039/0009; A61M 2039/229

(57) ABSTRACT

Embodiments herein relate to systems for generating suspensions/mixtures of therapeutic radioactive microspheres and carrier fluid. In an embodiment, a cancer therapy suspension generating system is included having a fluid container defining an interior volume, a fluid outlet out of the interior volume, an output line, and a coiled, looped, and/or folded fluid line. A first valve can be positioned between the fluid outlet and the fluid line while a second valve can be positioned between the fluid line and a distal portion of the output line. A pump can be included that pulls fluid out of the interior volume of the fluid container through the fluid outlet and first portion of the outlet line into the fluid line. Further, the pump pushes fluid out of the fluid line and into the second portion of the output line and onto a fluid delivery catheter. Other embodiments are also included herein.

This application claims the benefit of U.S. Provisional Application No. 63/673,064, filed Jul. 18, 2024, the content of which is herein incorporated by reference in its entirety.

### FIELD

Embodiments herein relate to systems with a space efficient tubing geometry for generating suspensions/mixtures of therapeutic radioactive microspheres and carrier fluid and delivering the same.

### BACKGROUND

According to the American Cancer Society, cancer accounts for nearly 25% of the deaths that occur in the United States each year. Cancerous tumors can form if one normal cell in any part of the body mutates and then begins to grow and multiply rapidly. Cancerous tumors can be a result of a genetic mutation to the cellular DNA or RNA that arises during cell division, an external stimulus such as ionizing or non-ionizing radiation, exposure to a carcinogen, or a result of a hereditary gene mutation. Regardless of the etiology, many cancerous tumors are the result of unchecked rapid cellular division.

Surgery is a common first-line therapy for many cancerous tumors. However, not every tumor can be surgically removed. Chemotherapy and immunotherapy are other common therapeutic approaches but can include substantial side effects. The use of radiation represents another approach. Specifically, radiation therapy aims at damaging the DNA of cancer cells so that they lose the capability to divide and proliferate, thus leading to the cell death process for the cancerous cells.

Brachytherapy is a form of radiation therapy where a sealed radiation source is placed inside or next to the area requiring treatment. As one form of brachytherapy, targeted radioembolization therapy can be used to treat unresectable tumors. For example, Y-90 glass microspheres can be delivered into or adjacent to a tumor through a microcatheter placed into an artery that supplies blood to the tumor. The beta radiation emitted by the Y-90 can exert a local radiotherapeutic effect on the tumor. Other radioisotopes can also be used in some types of brachytherapy.

### SUMMARY

Embodiments herein relate to systems with a space efficient tubing geometry for generating suspensions/mixtures of therapeutic radioactive microspheres and carrier fluid and delivering the same. In a first aspect, a cancer therapy suspension generating system is included having a fluid container defining an interior volume, a fluid outlet that passes out of the interior volume, a coiled, looped, and/or folded fluid line, an output line, a first valve positioned between the fluid outlet and the fluid line, a second valve positioned between the fluid line and a portion of the output line, and a pump. The pump pulls fluid out of the interior volume of the fluid container through the fluid outlet and into the fluid line when the first valve is in an open position and the second valve is in a closed position. The pump pushes fluid out of the fluid line and into the portion of the output line when the first valve is in a closed position and the second valve is in an open position.

In a second aspect, in addition to one or more of the preceding or following aspects, or in the alternative to some aspects, the fluid line includes a helical coil portion.

In a third aspect, in addition to one or more of the preceding or following aspects, or in the alternative to some aspects, the fluid line includes a folded portion.

In a fourth aspect, in addition to one or more of the preceding or following aspects, or in the alternative to some aspects, the fluid line includes a portion with a substantially circular perimeter.

In a fifth aspect, in addition to one or more of the preceding or following aspects, or in the alternative to some aspects, a vertical height of a coiled portion of the coiled fluid line can be less than 8 centimeters (3.15 inches).

In a sixth aspect, in addition to one or more of the preceding or following aspects, or in the alternative to some aspects, the coiled fluid line can have a length of at least about 4 feet (121.92 centimeters).

In a seventh aspect, in addition to one or more of the preceding or following aspects, or in the alternative to some aspects, the coiled fluid line can have an inner diameter of less than or equal to 0.040 inches (0.1016 centimeters).

In an eighth aspect, in addition to one or more of the preceding or following aspects, or in the alternative to some aspects, can further include a spindle, wherein a coiled portion of the coiled fluid line can be wrapped around the spindle.

In a ninth aspect, in addition to one or more of the preceding or following aspects, or in the alternative to some aspects, the pump can be a syringe pump or a piston pump.

In a tenth aspect, in addition to one or more of the preceding or following aspects, or in the alternative to some aspects, the cancer therapy suspension generating system can further include radioactive microspheres and a carrier fluid. The radioactive microspheres and the carrier fluid can be disposed within the interior volume.

In an eleventh aspect, in addition to one or more of the preceding or following aspects, or in the alternative to some aspects, the radioactive microspheres have a density of at least 1.4 times the density of the carrier fluid.

In a twelfth aspect, in addition to one or more of the preceding or following aspects, or in the alternative to some aspects, the fluid container can include a polymeric or glass vial.

In a thirteenth aspect, in addition to one or more of the preceding or following aspects, or in the alternative to some aspects, the interior volume can have a volume of 2 to 200 milliliters.

In a fourteenth aspect, in addition to one or more of the preceding or following aspects, or in the alternative to some aspects, the cancer therapy suspension generating system can further include a fluid inlet, wherein the fluid inlet can be configured to intake a gas to replace a volume of a fluid exiting the fluid outlet.

In a fifteenth aspect, a method of preparing a mixture/suspension for cancer therapy can be included. The method can include withdrawing a mixture or suspension of microspheres and carrier fluid from an interior volume of a fluid container and into a coiled, looped and/or folded fluid line using a pump, actuating a first valve to be in a closed position and a second valve to be in an open position, and expelling the mixture or suspension of microspheres and carrier fluid from the fluid line and into an output line with the pump.

In a sixteenth aspect, in addition to one or more of the preceding or following aspects, or in the alternative to some aspects, the fluid line includes a helical coil portion.

In a seventeenth aspect, in addition to one or more of the preceding or following aspects, or in the alternative to some aspects, the fluid line includes a folded portion.

In an eighteenth aspect, in addition to one or more of the preceding or following aspects, or in the alternative to some aspects, the fluid line includes a portion with a substantially circular perimeter.

In a nineteenth aspect, in addition to one or more of the preceding or following aspects, or in the alternative to some aspects, the pump can be a syringe pump or a piston pump.

In a twentieth aspect, in addition to one or more of the preceding or following aspects, or in the alternative to some aspects, the method can further include transferring the mixture or suspension of microspheres and carrier fluid from the output line to a fluid delivery catheter.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a cancer therapy suspension generating system in accordance with various embodiments herein.

FIG. 2 is a schematic view of a portion of a coiled fluid line in accordance with various embodiments herein.

FIG. 3 is a schematic side view of a portion of a coiled fluid line in accordance with various embodiments herein.

FIG. 4 is a schematic view of a portion of a coiled fluid line in accordance with various embodiments herein.

FIG. 5 is a schematic top view of a portion of a coiled fluid line in accordance with various embodiments herein.

FIG. 6 is a schematic view of portions of a cancer therapy suspension generating system in accordance with various embodiments herein.

FIG. 7 is a schematic view of portions of a cancer therapy suspension generating system is shown in accordance with various embodiments herein.

FIG. 8 is a schematic view of a portion of a coiled fluid line in accordance with various embodiments herein.

FIG. 9 is a schematic view of a cancer therapy suspension generating system in accordance with various embodiments herein.

FIG. 10 is a flowchart of a method in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

In some cancer therapy approaches including the use of radioactive microspheres or other particulates, the microspheres are delivered to the patient through a catheter or other delivery line, carried along by a flow of carrier fluid and, once in the body, the microspheres or other particulates and the carrier fluid mix with blood flow and then the resulting mixture passes onto a target site in the body. Before passing through the catheter, the carrier fluid is mixed with microspheres forming a mixture or suspension that is then conveyed to the catheter. Embodiments of cancer therapy suspension generating systems herein and components thereof can be used to generate mixtures/suspensions of radioactive microspheres and carrier fluid and deliver the same to a catheter, such as a microcatheter, which can be inserted into a patient and deliver the mixture or suspension into the patient.

In some cases, it may be desirable to withdraw only a portion of the total starting amount of microspheres within a fluid container for delivery to a patient at a given time. For example, perhaps there are multiple sites within the patient to be treated and each receives only a portion of the total starting amount of microspheres within the fluid container. This scenario can create challenges as it is important that the amount of microspheres per unit amount of carrier fluid remains consistent across multiple delivery operations and that the total amount of fluid withdrawn from the fluid container and delivered to the patient is accurate. Further, the highly dense microspheres used herein result in a substantial difference in the density of the microspheres versus the carrier fluid causing the microspheres to settle out of the mixture or suspension quickly in areas of low shear, which makes conveying the microspheres while achieving dosage consistency challenging.

However, embodiments of cancer therapy suspension generating systems herein can withdraw portions of a total volume of microspheres and carrier fluid from a fluid container while maintaining dosage consistency. Embodiments herein can withdraw a desired dosage amount of microspheres and carrier fluid from a fluid container and then temporarily hold the same in a coiled, folded, and/or looped fluid line component before subsequent delivery to the patient. The coiled, folded, and/or looped fluid line can hold a desired dosage amount of microspheres and carrier fluid. When fluid flow temporarily stops in the tubing, such as after the microspheres and carrier fluid have been withdrawn from the fluid container, the microspheres will generally fall out of suspension and lay on the bottom of the tubing. The use of a small diameter tubing is advantageous in that spheres will readily re-suspend when the fluid flow resumes. In contrast, for larger diameter tubing the fluid movement will be slower for a given volumetric flow rate and the wall shear stress of the moving fluid will be lower making it harder to resuspend the microspheres in the carrier fluid. As such, inner diameters of tubing herein, such as the tubing used for the coiled, looped, or folded portion, can have a small inner diameter, such as 0.040 inches or less (1.01 millimeters or less), or in some cases 0.020 inches or less (0.505 millimeters or less).

However, as a result of the small inner diameter, typically a very long length of fluid line is required to hold the desired dosage amount of microspheres and carrier fluid. For example, at such small inner diameters, the fluid line might have to be 3, 4, 5, 6, 7, 8, 10, 12, 15, or even 20 feet or longer (91.44, 121.92, 152.4, 182.88, 213.36, 243.84, 304.8, 365.76, 457.2, or 609.6 centimeters) to accommodate desired dosages. However, the use of a very long fluid line can be quite inconvenient in the clinical environment as it would take up a lot of space and would be difficult to manage without becoming kinked or getting in the way. As such, in embodiments herein, a portion of the fluid line can be configured in a space efficient manner such that the fluid line is coiled or otherwise looped back, folded, or bent back upon itself and takes up only limited surface space versus a straight fluid line of equal length and can be much more convenient for use by clinicians. References in the description herein to coiled fluid line portions shall also be inclusive of folded or looped fluid line portions unless the context dictates otherwise.

The term “suspension” as used herein in reference to what embodiments of systems herein form and/or deliver as therapy to patients including carrier fluid and radioactive microspheres shall be used interchangeably with the term “mixture” unless the context dictates otherwise.

In an embodiment, a cancer therapy suspension generating system can be included having a fluid container defining an interior volume, a fluid outlet passing out of the interior volume, an output line, and a coiled fluid line. A first valve can be positioned between one portion of the fluid outlet and the coiled fluid line while a second valve can be positioned between the coiled fluid line and another portion of the output line. A pump can be included that pulls fluid out of the interior volume of the fluid container through the fluid outlet and first portion of the outlet line into the coiled fluid line when the first valve is in an open position and the second valve is in a closed position. Further, the pump pushes fluid out of the coiled fluid line and into the second portion of the output line (and onto a fluid delivery catheter) when the first valve is in a closed position and the second valve is in an open position.

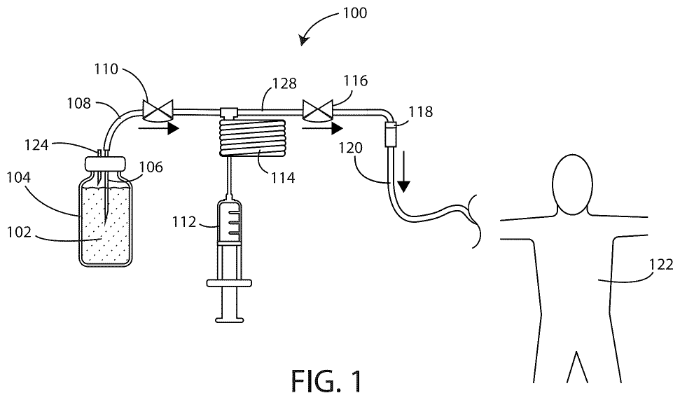

Referring now to FIG. 1, a schematic view of a cancer therapy suspension generating system 100 is shown in accordance with various embodiments herein. The cancer therapy suspension generating system 100 includes a mixture or suspension of radioactive microspheres and carrier fluid 102 (or “microsphere suspension”) within a fluid container 104. In some embodiments, the carrier fluid can be a saline solution, however other carrier fluids can also be used herein. Details regarding exemplary microspheres are provided in greater detail below. However, in many embodiments the microspheres are much more dense than the carrier fluid and can have a density that is multiples of the carrier fluid, such as 1.4, 1.8, 2, or 3 times or more than the carrier fluid. The fluid container 104 can take the form of a polymeric or glass vial, however other materials can also be used for the fluid container 104. In various embodiments the interior volume of the fluid container can be about 2 milliliters to about 200 milliliters or more.

The system 100 also includes a fluid outlet 106 from the fluid container 104 connecting to a first portion 108 of an output line. The system also includes a second portion 128 of the output line. The passage of the microsphere suspension through the output line can be controlled by a first valve 110 and a second valve 116. A coiled fluid line 114 can be in fluid communication with both the first portion 108 of output line and the second portion 128 of output line at a point between the first valve 110 and the second valve 116. As such, the first valve 110 can be positioned between a fluid outlet 106 and a coiled fluid line 114 and the second valve 116 can be positioned between a coiled fluid line 114 and a distal connector 118. The system 100 can also include a pump 112 to pull fluid out of an interior volume of a fluid container 104 through the fluid outlet 106 and into the coiled fluid line 114, such as when the first valve 110 is in an open position and the second valve 116 is in a closed position. In various embodiments, the pump 112 can be a syringe pump 112 or piston pump, however other types of pumps are also contemplated herein. The pump 112 can also be used to push the microsphere suspension out of the coiled fluid line 114, into the second portion 128 of output line and towards distal connector 118 and a microcatheter 120 such as when the first valve 110 is closed and the second valve 116 is open. The microcatheter 120 can be positioned within a patient 122 to facilitate delivery of the microsphere suspension thereto. In various embodiments, the system can further include a fluid inlet 124 for the fluid container 104, wherein the fluid inlet 124 can be configured to intake a gas to replace a volume of a carrier fluid exiting the fluid outlet 106. In some embodiments, the fluid inlet 124 can be configured to intake a liquid, such as additional carrier fluid, to replace the volume of carrier fluid exiting the fluid outlet 106.

As referenced above, the small inner diameter of the fluid line used results in a relatively long length being required in order to hold a desired dosage amount of microspheres and carrier fluid. However, fluid lines herein can include a coiled, looped and/or folded portion such that it takes up only a limited amount space (e.g., is space efficient) and can be convenient for use by clinicians.

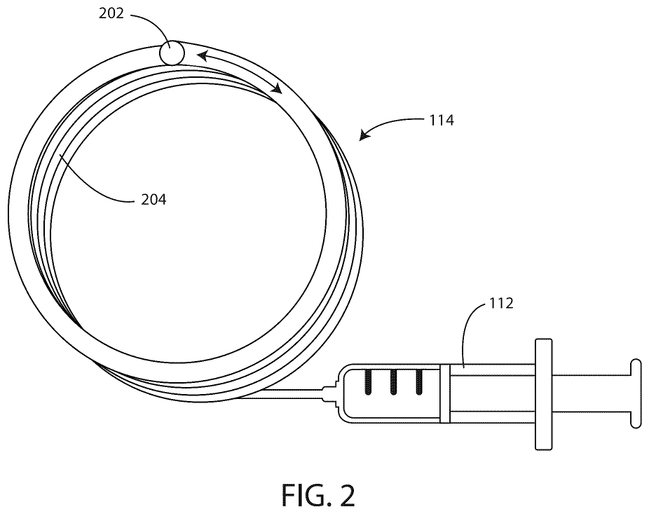

Referring now to FIG. 2, a schematic view of a portion of a coiled fluid line 114 is shown in accordance with various embodiments herein. As before, the cancer therapy suspension generating system includes a pump 112. The cancer therapy suspension generating system also includes a coiled fluid line 114 connected to the pump 112. As shown, the coiled fluid line 114 includes a coil inlet 202 which may, for example, connect to the output line and/or a junction in the same, such as a junction between the first portion and the second portion of the output line.

The coiled fluid line 114 can be formed of various materials including, but not limited to, polyurethane, polyvinylchloride, polypropylene, silicone, or the like. As referenced above, the fluid line can have a relatively small inner diameter. In some embodiments, the fluid line can have an inner diameter of 0.1, 0.075, 0.050, 0.040, 0.035, 0.030, 0.025, or 0.020 inches or less (2.54, 1.905, 1.27, 1.01, 0.89, 0.76, 0.63, 0.51 millimeters or less), or can have an inner diameter falling within a range between any of the foregoing.

The coiled fluid line 114 includes a coil portion 204 which can take various forms. In general, the coil portion 204 includes a plurality of passes or sections of the fluid line. The number of passes can vary based on the length of the fluid line as well as the diameter of the coil, however, in various embodiments, the number of passes can be from 2 to 25 or more. In some embodiments, the coil portion 204 can specifically take the form of a helical coil portion 204. When viewed from the top, the coil portion 204 can include an outer perimeter that is substantially circular. However, the outer perimeter can also take on other shapes such as ovoid, rounded polygonal, irregular, or the like. In some embodiments, a circular shape can be advantageous for integrating the coil with a sensor such as a radiation sensor, that can be used to assess the quantity or radioactive intensity of the microspheres or isotope inside the coil. With a circular shape, the tubing can all be at an equal distance from a cylindrical or linear shaped sensor.

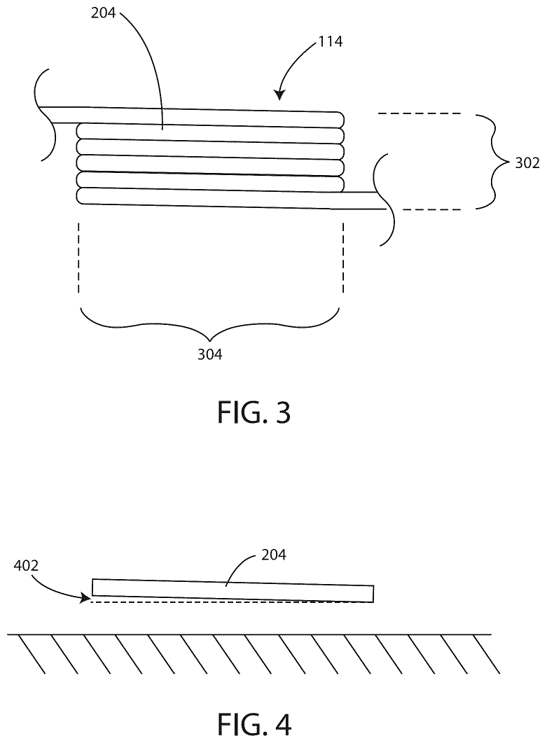

In some embodiments the plurality of passes or sections of the coil portion 204 can overlap one another such that there is a vertical dimension to the coil. Referring now to FIG. 3, a schematic side view is shown of a portion of a coiled fluid line 114 and specifically a coil portion 204 thereof in accordance with various embodiments herein. The coil portion is shown with a height 302 and a diameter 304. While not intending to be bound by theory, it can be advantageous to keep the height 302 as small as possible. This is at least because the height 302 can be related to a degree of slope (with respect to the direction of gravity) of the overlapping passes of the fluid line, with (all things being equal) greater height resulting in greater slope. The more slope, the greater the impact that gravity has on the dense spheres undesirably moving through the fluid line. In various embodiments, the height 302 of the coil portion 204 can be less than or equal to about 8, 7, 6, 5, 4, 3, 2, or even 1 centimeter (3.15, 2.76, 2.36, 1.96, 1.57, 1.18, 0.78, 0.39 inches). In various embodiments, the diameter 304 of the coil portion 204 can be from about 1 centimeter to about 20 centimeters or more (0.39 inches to 7.87 inches).

Referring now to FIG. 4, a schematic view of a portion of a coiled fluid line is shown in accordance with various embodiments herein. For clarity, FIG. 4 only depicts a portion of a single pass or segment of the coil portion 204. As can be seen, the coiled fluid line includes an incline angle 402 or slope with respect to gravity. In various embodiments herein, the incline angle 402 can be less than about 10, 8, 5, 4, 3, 2, or even 1 degree, or can be within a range between any of the foregoing.

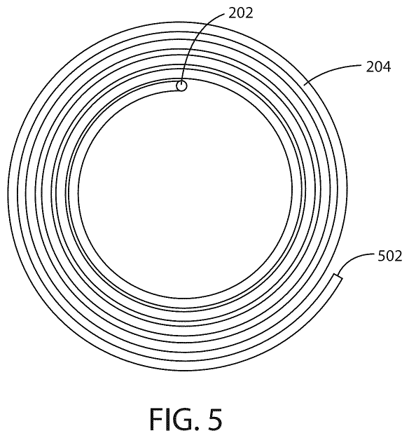

It will be appreciated the coil portion herein can take on various specific shapes. As described above, in some embodiments the coil portion can be helical. However, in some embodiments the coil portion can also be at least partly spiral. Referring now to FIG. 5, a schematic top view of a portion of a coiled fluid line including a coiled portion 204 is shown in accordance with various embodiments herein. The coiled portion 204 includes a coil inlet 202 as well as a coil outlet 502. In various embodiments, the coiled fluid line 114 is configured as a spiral with successive passes or sections being of a different diameter than previous passes or sections. It will be appreciated that, in some embodiments, certain shape features herein can be combined. By way of example, in some embodiments the coil portion can be both helical and spiral.

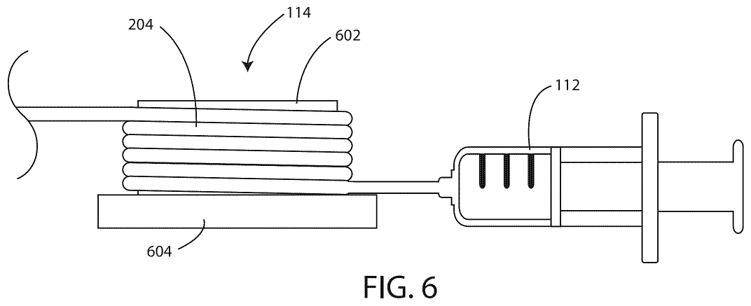

In some embodiments, other components can be included to maintain the coiled portion of the coiled fluid line in a particular configuration or shape. Referring now to FIG. 6, a schematic view of portions of a cancer therapy suspension generating system is shown in accordance with various embodiments herein. A coiled fluid line 114 and a coil portion 204 thereof is shown connected to a pump 112. In this embodiment, the cancer therapy suspension generating system includes a spindle 602. The spindle 602 can take the form of a rod and can optionally include a base 604. The coil portion 204 can be wrapped around the spindle 602 and/or attached thereto and thereby the shape of the coil portion 204 can be maintained.

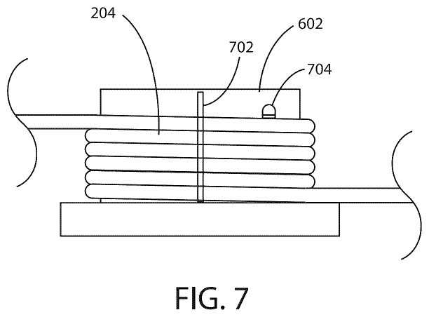

In some embodiments, the spindle can include indicia thereon to facilitate measurement of fluid volumes within the coiled fluid line. Referring now to FIG. 7, a schematic view is shown of portions of a cancer therapy suspension generating system in accordance with various embodiments herein. FIG. 7 specifically shows a coil portion 204 wrapped around a spindle 602. The fluid line can be substantially transparent such that fluid therein can be seen or at least a liquid/gas boundary therein can be seen. In some embodiments, indicia 702 or visual markers can be disposed on the spindle 602 which can also be seen through the transparent fluid line and thus the indicia 702 can be visible underneath the fluid line that is wrapped over the spindle 602. In this manner, the position of the fluid within the fluid line (and/or the position of a liquid/gas boundary) can be accurately determined relative to the spindle 602 allowing for more accurate and repeatable operations, such as more accurate and repeatable amounts of the therapeutic suspension being drawn into the coiled fluid line. In the alternative or in addition, in some embodiments, indicia or visual markers can be disposed on the fluid line itself.

In some embodiments, the system and/or the spindle 602 thereof can include one or more sensors to quantify an amount of or an intensity of the fluid, microspheres, or combinations of fluid and microspheres within the fluid line. In some embodiments, the system and/or the spindle thereof can include one or more sensors to detect air bubbles in the fluid. The sensor(s) can include one or more of optical sensors, ultrasonic fluid sensors, ultrasonic bubble sensors, thermal flow sensors, pressure sensors, and the like. In some embodiments, the system and/or the spindle thereof can include one or more radiation sensors (such as gas-filled radiation detectors including Geiger-Mueller tube sensors, ionization chamber sensors, and proportional counters, a scintillation type sensor, a solid state radiation sensor, or the like) that can be used to assess the quantity of microspheres or isotope within the fluid line and/or activity of the same. FIG. 7 shows a sensor 704, which could be any of these types of sensors. The sensor 704 is mounted on the spindle 602 in a position to interface with the coiled portion 204 of the fluid line, but could be mounted at other locations and can be entirely separate from a spindle 602. For example, in some embodiments, one or more sensors can be mounted on or adjacent to the outlet line of the system. In some embodiments, a sensor itself can effectively act as a spindle. For example, the fluid line can be wrapped around a sensor.

As described above, in some embodiments, the system can include a fluid line portion that is coiled or otherwise looped back, folded, or bent back upon itself such that it takes up only limited surface space versus a straight fluid line of equal length and can be convenient for use by clinicians. Beyond a coil shape, a portion of the fluid line can be configured in a flat-plane folded arrangement with a radius on the folds sufficient to avoid kinking the tubing. A circle or any shape that loops back around 360 degrees (such as a shape with four 90 degree turns or a shape with two 180 degree turns) can be described as having a sum of internal angles of 360 degrees. Embodiments herein of coiled, looped, folded, or bent fluid lines can loop around multiple times and thus have a sum total of internal angles equaling a plurality of 360 degrees turns or more, such as 720, 1080, 1440, 1800, 3600, 7200 degrees or more, or an amount falling within a range between any of the foregoing.

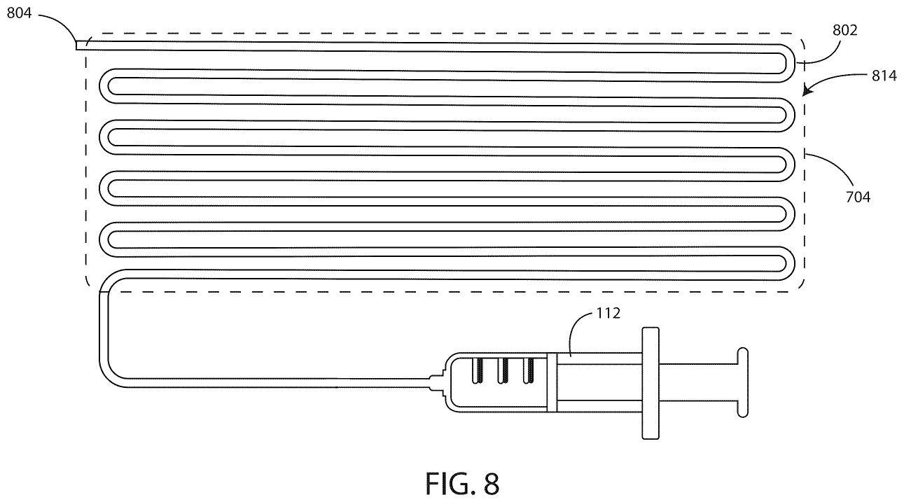

Referring now to FIG. 8, a schematic view is shown of a portion of a fluid line in accordance with various embodiments herein. Specifically, FIG. 8 shows a portion of a fluid line 814 with a series of folds 804 (or turns, such as 180 degree turns) and substantially straight portions between folds 804, a pump 112, and an inlet 802. The fluid line 814 in this example can be arranged on a flat-plane as a folded arrangement or zig-zag pattern with a radius on the folds 804 sufficient to avoid kinking the tubing. In some embodiments, a sensor 704 (such as one of the types previously discussed) can be configured in a flat-plane format and can be configured to interface with the flat-plane folded fluid line 814 to detect aspects such as radiation of microspheres within the flat-plane folded fluid line 814.

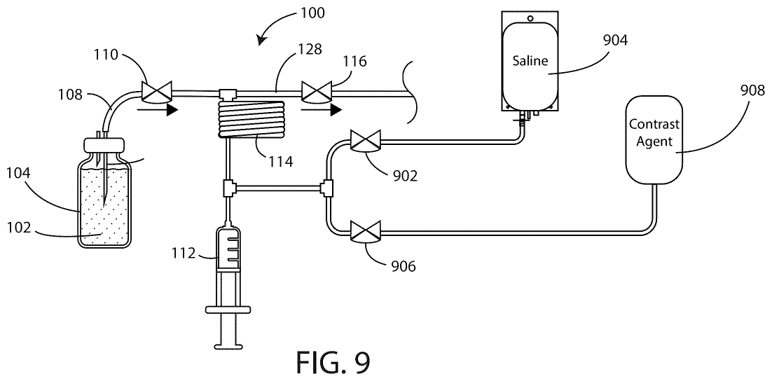

In some embodiments, the system can include additional components compared with that shown in FIG. 1. Referring now to FIG. 9, a schematic view of a cancer therapy suspension generating system 100 is shown in accordance with various embodiments herein. As before, a mixture or suspension of radioactive microspheres and carrier fluid 102 is disposed within a fluid container 104. A first portion 108 of an output line passes through a first valve 110. The cancer therapy suspension generating system 100 also includes a pump 112 and a coiled fluid line 114. A second portion 128 of the output line passes through second valve 116.

In this embodiment, the cancer therapy suspension generating system 100 also includes a third valve 902 and a fourth valve 906. The third valve 902 provides selective fluid communication with a source of fluid, such as a saline bag 904 or reservoir. The fourth valve 906 provides selective fluid communication with another fluid that may be used such as a contrast agent 908 reservoir. By actuating the valves appropriately, the pump 112 can be used to withdraw a fluid from either the saline bag 904 or the contrast agent 908 reservoir and push the same through the coiled fluid line 114 and through the second portion 128 of the output line and onto the fluid delivery catheter and the patient.

### Methods

Many different methods are contemplated herein, including, but not limited to, methods of making and/or delivering suspensions/mixtures of microspheres and carrier fluid for cancer therapy, methods of using suspensions/mixtures of microspheres and carrier fluid for cancer therapy, and the like. Aspects of system/device operation described elsewhere herein can be performed as operations of one or more methods in accordance with various embodiments herein.

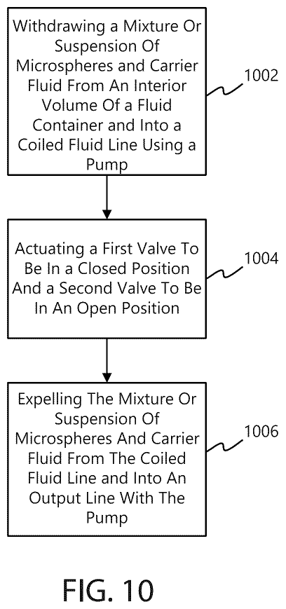

Referring now to FIG. 10, a flowchart of a method in accordance with various embodiments herein is shown. In specific, major operations of a method of preparing a mixture/suspension for cancer therapy is shown. The method can include an operation of withdrawing a mixture or suspension of microspheres and carrier fluid from an interior volume of a fluid container and into a coiled fluid line using a pump 1002. The method can further include an operation of actuating a first valve to be in a closed position and a second valve to be in an open position 1004. The method can further include an operation of expelling the mixture or suspension of microspheres and carrier fluid from the coiled fluid line and into an output line with the pump 1006.

In an embodiment of the method, the coiled fluid line includes a helical coil portion. In an embodiment of the method, the coiled fluid line includes a spiral portion. In an embodiment of the method, the coiled fluid line includes a portion with a substantially circular perimeter. In an embodiment of the method, the pump is a syringe pump.

In an embodiment, the method can further include transferring the mixture or suspension of microspheres and carrier fluid from the output line to a fluid delivery catheter.

### Microspheres

Microspheres herein can include those with a combination of yttria, alumina, and silica. By way of example, in some embodiments, microspheres herein can include Y₂O₃—Al₂O₃—SiO₂ in a 40:20:40 wt. % ratio. It will be appreciated however, that other types of microspheres are also contemplated herein including those with other materials.

In some embodiments, microspheres can be prepared by combining yittrium-89 with alumina and silica, in some cases also using a flame spheroidization method, and using neutron bombardment to convert Y-89 into the beta emitting radioisotope Y-90. In various embodiments, the amount of beta radiation can exceed 400, 600, 800, 1000, 1500, 2000, 2500, 3000, 4000, 5000, 6000, 7000, 8000, 9000, 10000, or even 12000 Becquerel (Bq) per sphere or higher at the time of activity calibration (recognizing that the amount of radiation will drop after that point as the Y-90 radioisotope decays). In some embodiments, the microspheres can be provided in a vial with activity of 3 Gigabecquerel (GBq) or lower up to 20 GBq or higher (at calibration time or “reference date and time”). However, in some embodiments, the microspheres can be provided in a vial with activity of less than 3, 2.75, 2.5, 2.25, 2, 1.75, 1.5, 1.25, 1.0, 0.75, 0.5, 0.4, 0.3, 0.35, 0.2, 0.15, 0.1, 0.05, or 0.01 GBq, or less at calibration time, or an amount falling within a range between any of the foregoing. It will be appreciated that the use of other isotopes is also contemplated herein.

It will be appreciated that dosages can vary based on factors including the type of tumor/tissue to be treated, location of the tumor/tissue to be treated, factors specific to a particular patient, and the like. In some embodiments the dosage of the therapy can be less than or equal to 5000 Gray (Gy), 4500 Gy, 4000 Gy, 3500 Gy, 3000 Gy, 2500 Gy, 2000 Gy, 1500 Gy, 100 Gy, 500 Gy, 400 Gy, 300 Gy, 250 Gy, 225 Gy, 200 Gy, 180 Gy, 150 Gy, 120 Gy, 100 Gy, 90 Gy, 80 Gy, 70 Gy, 60 Gy, 50 Gy, 40 Gy, 30 Gy, or 20 Gy, or an amount falling within a range between any of the foregoing.

The size of the microspheres can be extremely small. In some embodiments, the average diameter of the microspheres can be from about 15 micrometers (μm) to about 35 μm. However, in some embodiments the microspheres can be somewhat smaller or larger.

The density of the microspheres can be quite high. In some embodiments, the density of the microspheres can be above 1.4, 1.8, 2, 2.5, or 3 grams/milliliter (g/mL), such as from 3.1 to 3.5 g/mL, or about 3.3 g/mL. By comparison, the density of water at room temperature is about 0.9978 g/mL. As such, the density of microspheres is much higher than an exemplary carrier fluid such as a saline solution which influences how readily such microspheres can settle out of a suspension.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A cancer therapy suspension generating system comprising:
a fluid container, the fluid container defining an interior volume;
a fluid outlet, wherein the fluid outlet passes out of the interior volume;
a coiled, looped, and/or folded fluid line;
an output line;
a first valve, wherein the first valve is positioned between the fluid outlet and the fluid line;
a second valve, wherein the second valve is positioned between the fluid line and a portion of the output line;
a pump;
wherein the pump pulls fluid out of the interior volume of the fluid container through the fluid outlet and into the fluid line when the first valve is an open position and the second valve is in a closed position; and
wherein the pump pushes fluid out of the fluid line and into the portion of the output line when the first valve is in a closed position and the second valve is in an open position.

2. The cancer therapy suspension generating system of claim 1, wherein the fluid line includes a helical coil portion.

3. The cancer therapy suspension generating system of claim 1, wherein the fluid line includes a folded portion.

4. The cancer therapy suspension generating system of claim 1, wherein the fluid line includes a portion with a substantially circular perimeter.

5. The cancer therapy suspension generating system of claim 1, wherein a vertical height of a coiled portion of the fluid line is less than 8 centimeters (3.15 inches).

6. The cancer therapy suspension generating system of claim 1, wherein the fluid line has a length of at least about 4 feet (121.92 centimeters).

7. The cancer therapy suspension generating system of claim 1, wherein the fluid line has an inner diameter of less than or equal to 0.040 inches (0.1016 centimeters).

8. The cancer therapy suspension generating system of claim 1, further comprising a spindle, wherein a coiled portion of the fluid line is wrapped around the spindle.

9. The cancer therapy suspension generating system of claim 1, wherein the pump is a syringe pump or a piston pump.

10. The cancer therapy suspension generating system of claim 1, further comprising:
radioactive microspheres, wherein the radioactive microspheres are disposed within the interior volume; and
a carrier fluid, wherein the carrier fluid is disposed within the interior volume.

11. The cancer therapy suspension generating system of claim 10, wherein the radioactive microspheres have a density of at least 1.4 times the density of the carrier fluid.

12. The cancer therapy suspension generating system of claim 1, the fluid container comprising a polymeric or glass vial.

13. The cancer therapy suspension generating system of claim 1, wherein the interior volume has a volume of 2 to 200 milliliters.

14. The cancer therapy suspension generating system of claim 1, further comprising a fluid inlet, wherein the fluid inlet is configured to intake a gas to replace a volume of a fluid exiting the fluid outlet.

15. A method of preparing a mixture/suspension for cancer therapy comprising:
withdrawing a mixture or suspension of microspheres and carrier fluid from an interior volume of a fluid container and into a coiled, looped and/or folded fluid line using a pump;
actuating a first valve to be in a closed position and a second valve to be in an open position; and
expelling the mixture or suspension of microspheres and carrier fluid from the fluid line and into an output line with the pump.

16. The method of preparing a mixture/suspension for cancer therapy of claim 15, wherein the fluid line includes a helical coil portion.

17. The method of preparing a mixture/suspension for cancer therapy of claim 15, wherein the fluid line includes a folded portion.

18. The method of preparing a mixture/suspension for cancer therapy of claim 15, wherein the fluid line includes a portion with a substantially circular perimeter.

19. The method of preparing a mixture/suspension for cancer therapy of claim 15, wherein the pump is a syringe pump or a piston pump.

20. The method of preparing a mixture/suspension for cancer therapy of claim 15, further comprising transferring the mixture or suspension of microspheres and carrier fluid from the output line to a fluid delivery catheter.
